(19) United States
(12) Patent Application Publication
(10) Pub. No.: US 2014/0343371 A1
(43) Pub. Date: Nov. 20, 2014

(54) WEARABLE SENSOR DEVICE FOR HEALTH MONITORING AND METHODS OF USE

(71) Applicants: THOMAS SKERIK SOWERS, II, Chapel Hill, NC (US); JOHN F. VOLKMAR, Boulder, CO (US)

(72) Inventors: THOMAS SKERIK SOWERS, II, Chapel Hill, NC (US); JOHN F. VOLKMAR, Boulder, CO (US)

(21) Appl. No.: 14/277,013

(22) Filed: May 13, 2014

Related U.S. Application Data
(60) Provisional application No. 61/823,383, filed on May 14, 2013.

(51) Int. Cl. A61B 5/1455; A61B 5/024; A61B 5/021; A61B 5/0205

(52) U.S. Cl. CPC A61B 5/1455; A61B 5/0205; A61B 5/02427; A61B 5/02141; A61B 5/14552
USPC 600/301; 600/322; 600/479; 600/480; 600/323

(57) ABSTRACT

A device for monitoring health information. The device comprises a material capable of being worn on a person's body, an optical generator, an optical sensor, a processor, and an interface for communicating with an external device. The device may also include two EKG sensors. The device non-invasively monitors a variety of health characteristics and transmits the health data to a base station such as a smartphone. The base station includes a software application that can display, store, and analyze the user's health data. The user may review his health information on the base station's display. In an alternative embodiment, the base station transmits the health data to a secure remote server. The user may review his health information on a website associated with the remote server.

### CROSS REFERENCE TO RELATED APPLICATIONS

The present application claims the benefit of provisional patent application Ser. No. 61/823,383, filed on May 14, 2013.

### BACKGROUND OF THE INVENTION

The present invention relates generally to non-invasively monitoring health characteristics, and, more particularly, to methods and apparatus for non-invasively monitoring a person's heath characteristics, including but not limited to vital signs, using optical generators and sensors.

There are many known methods and apparatus for non-invasively measuring a person's health characteristics. For example, pulse oximeters are well-known in the art. A pulse oximeter measures the difference between a person's levels of oxyhemoglobin and deoxyhemoglobin in order to determine the oxygen saturation of a person's blood. The pulse oximeter has two light-emitting diodes—one that sends out (invisible) infrared light and one that sends out red light. The light from the two wavelengths is passed through the user to a photo-detector. The infrared light-emitting diode (“LED”) is used as a baseline and compared to the red LED, which has some of its intensity absorbed by a person's red blood. Thus, the difference between the two colors gives a measure of what fraction of the hemoglobin in the person's blood is oxygenated. Deoxygenated blood absorbs light differently than oxygenated blood. Therefore, the ratio of the difference of absorption at is an indication of blood oxygenation. A well-designed pulse oximeter will continuously look at the ratio of these two wavelengths. The phototransistor acts like a variable resistor which conducts different amounts of current depending on how much light it sees. This changes the voltage in a way that changes with heartbeats.

Electrocardiograms (“ECGs” or “EKGs”) are also well-known in the art. EKGs measure the heart's electrical activity. To form a heartbeat, the SA node generates an electrical shock, which stimulates the atria to contract. The AV node, in combination with the bundle of His, control contraction of the ventricles. An ECG measures these voltage changes on the surface of the skin. Heart rate monitors are also well-known in the art. They may be part of an EKG or they may be a separate apparatus. Likewise, accelerometers are well-known in the art.

There is a concern that the current systems for measuring a person's health characteristics, such as the devices described above, cannot easily and accurately be measured on a single device. Furthermore, there is a concern that the current systems for measuring a person's health characteristics cannot be measured in a continuous, efficient manner so as to provide a user with notice when his health characteristics indicate a potential problem.

Therefore, there is a need in the art for a single apparatus to non-invasively measure a variety of health characteristics, as well as methods for using the apparatus to provide continuous monitoring, which overcome the shortcomings of the prior art.

### BRIEF SUMMARY OF THE INVENTION

The present invention is intended to provide a health monitoring device to non-invasively monitor a variety of health characteristics. The invention allows a user to conduct low-cost, comprehensive, continuous monitoring of his health information. The health monitoring device may be a wrist band, or any similar embodiment. The device contains an optical generator, an optical sensor, a wireless communication device for transmitting data from the device, and a processor that can execute programs or process data. The device may also include two ECG/EKG sensors on the external portion of the device.

When the device is worn by a user, the optical generator and the optical sensor are placed within close proximity of the skin. The optical generator emits wavelengths of light. The optical sensor uses the wavelengths of light generated by the optical generator to gather data from the user's blood characteristics. In another embodiment, the user applies his thumbs to both ECG/EKG sensor, thereby completing a circuit. The data collected by the optical sensor and/or ECG/EKG sensors is digitized into a time-dependent optical waveform, which is then transmitted to a base station, such as a smartphone.

The base station collects and synchronizes data from the device at predetermined intervals. The base station downloads the data to a software application, such as a mobile application, located on the base station. The software application may combine the received data with current environmental data collected by the base station and/or the user may input additional data pertaining to health (such as historical and/or current health data) directly into the software application.

The software application analyzes the data and generates health information. The generated health information may relate to, but not limited to: albumin levels; proteins; glucose; the blood's bicarbonate level; red blood cells; white blood cells; the user's hemoglobin concentration; blood volume; blood oxygenation levels; pulse rate (PR); blood pressure; saturation of peripheral oxygen (spO2); characteristics of hemoglobin (including but not limited to dehydration or hyperhydration; nitric oxide; anemia; carboxyhemoglobin; methemoglobin; oxygen content; oxygen saturation); the user's heart's electrical activity; the user's heartbeat; the user's risk of heart attack; pericarditis, angina; palpitations; thickness of the user's heart chambers' walls; the user's risk of stroke; sleep apnea; hypertension; the user's risk of congestive heart failure; atrial fibrillation; systolic embolism; the user's speed of movement; physiological changes during exercise (to include hydration and salt levels); the user's blood alcohol content; weight; body fat; lean mass; muscle mass; bone mass; body water; daily caloric intake; BMI; and visceral fat rating.

The base station pushes the data over a computer network to a secure remote server. The remote server will collect, retain, store and display both historical and current health data from the device. The data is stored in a secure environment, with security levels equal to those of online banking and other confidential information. The user may input additional data pertaining to health (such as historical and/or current health data) directly into a website on the remote server. The software application and/or website may generate a warning or alert if the user's health information reflects a dangerous condition. In another embodiment, the health information on the remote server may be monitored by an authorized call center.

### BRIEF DESCRIPTION OF THE DRAWINGS

FIG. 1 is a plan view of an embodiment of a wearable sensor device.

FIG. 2 illustrates two isometric views of an embodiment of the wearable sensor device.

FIG. 3 is a flow chart of a method of using a wearable sensor device for health monitoring.

### DETAILED DESCRIPTION OF THE INVENTION

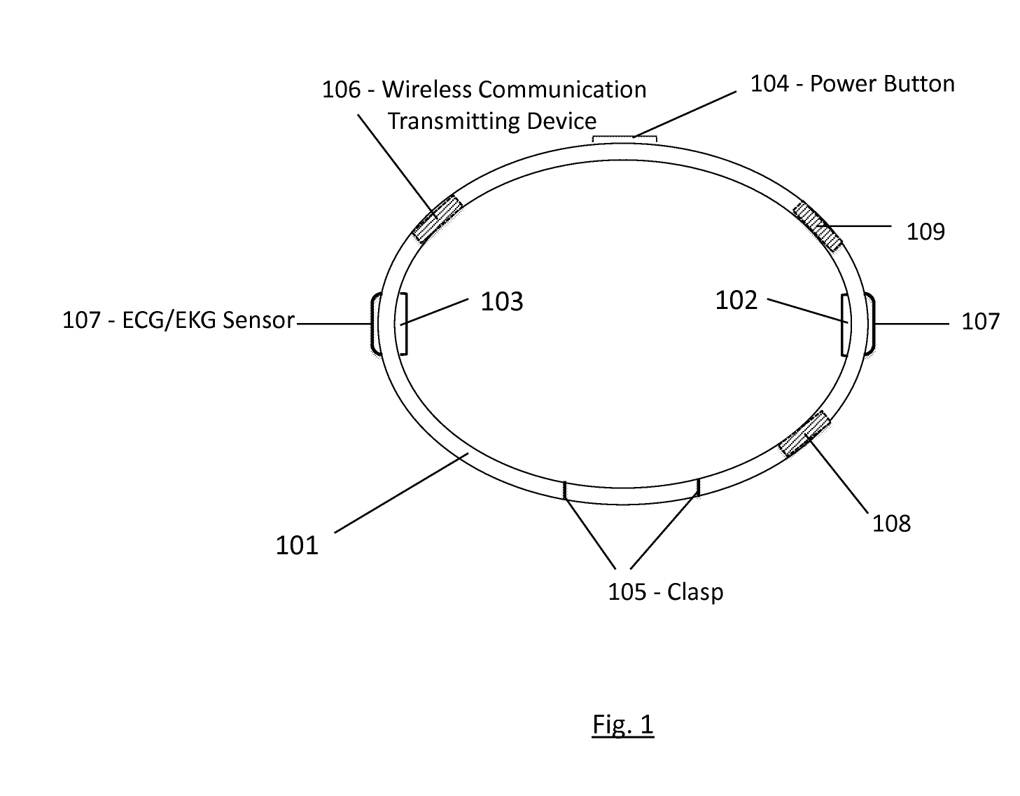

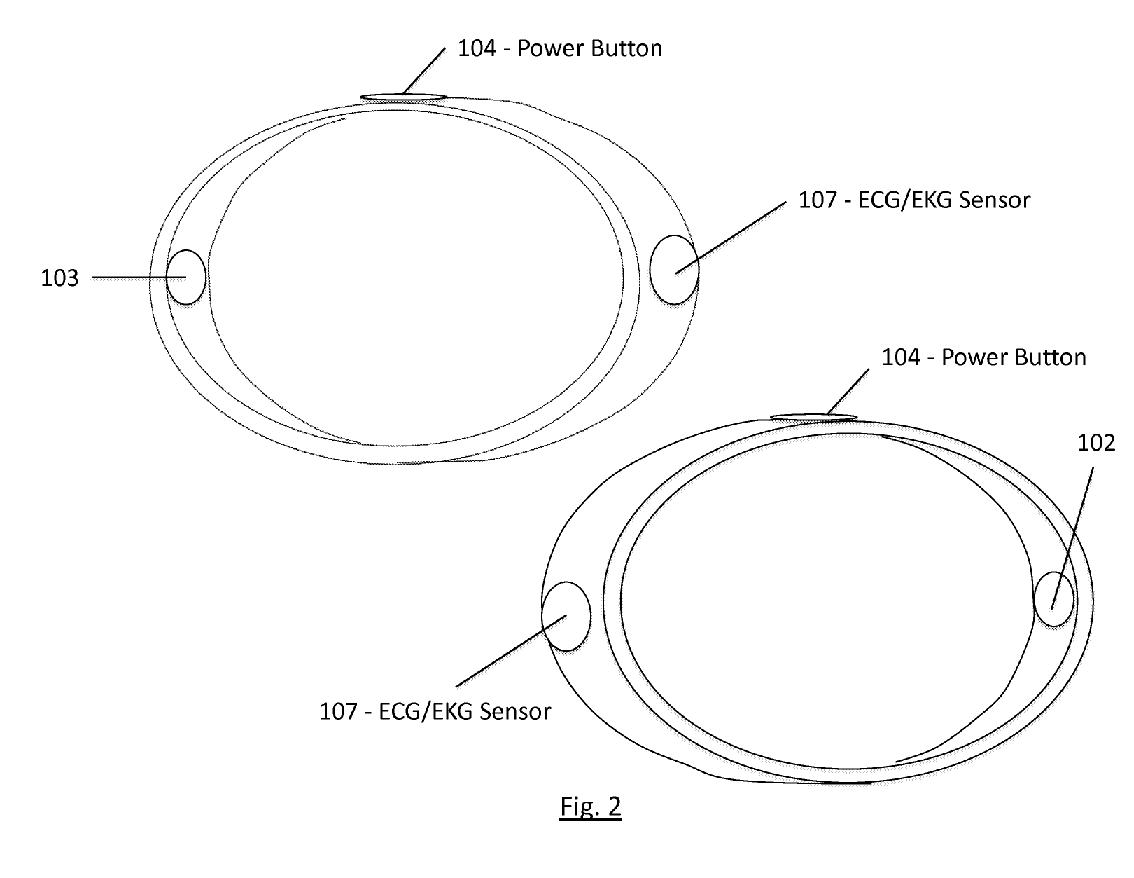

Referring now to the drawings and the illustrative embodiments depicted therein, and specifically FIG. 1 and FIG. 2, a health monitoring device (“the device”) 101 may non-invasively monitor a variety of health characteristics, including heart rate, temperature, EKG measurements and blood characteristics. The device 101 is shown as a wrist band, but may be any similar embodiment such as a ring, a belt, a band, a hat band, socks, or an implantable device, or attachable to a watch. The device 101 may be waterproof or water-resistant. The device 101 may be made of any plastic, metal, rubber or any other materials known in the art. The internal side of the device 101 contains one or more optical generators 102 and one or more optical sensors 103. The optical generator 102 may be an LED. To prevent burning or irritation to the skin, the optical generator 102 may be at a low duty rate of modulation. The optical sensor 103 may be a photodetector, such as a photodiode or phototransistor. The one or more optical generators 102 are placed on an opposite side of the device 101 from the one or more optical sensors 103. Thus, when the device 101 is worn as a wrist band, the one or more optical generators 102 are on the opposite side of the wrist from the one or more optical sensors 103. The device 101 contains a power button 104 to turn the device 101 on and off. In one embodiment, the device 101 contains connecting clasp 105. In another embodiment, the device 101 does not contain a connecting clasp 105. The device 101 contains a wireless communication device 106, such as an infrared LED transmitter, for transmitting data from the device 101. The device 101 includes two ECG/EKG sensors 107 on the external portion of the device 101.

The device 101 contains a long duration rechargeable battery 108 located inside the device's outer casing. The battery 108 may be recharged without having to remove the battery from the device 101. The battery 108 may be recharged by any standard method of recharging batteries, including but not limited to connecting the device 101 to a charger such as an AC adapter or recharging the device through solar power or bodily movement. The device 101 contains a processor 109 and may contain a computer memory. The processor 109 can be any type of device capable of processing electronic instructions including microprocessors, microcontrollers, host processors, controllers, and application specific integrated circuits (ASICs). The processor 109 can execute programs or process data. The device 101 may optionally contain an accelerometer. The device 101 may also contain a global positioning system (GPS) receiver that relays a location (e.g., latitude, longitude, and altitude) of the device.

The device 101 may also perform a user's ECG/EKG measurements. The device 101 may also measure a user's body temperature and a user's movement. In another embodiment, the device 101 includes a mouthpiece into which the user may breathe. In another embodiment, the optical sensor 103 non-invasively collects data based on the user's sweat and the device 110 includes a pH sensitive dye which changes color when the pH of the sweat changes, as detected with a diffuse reflectance detector.

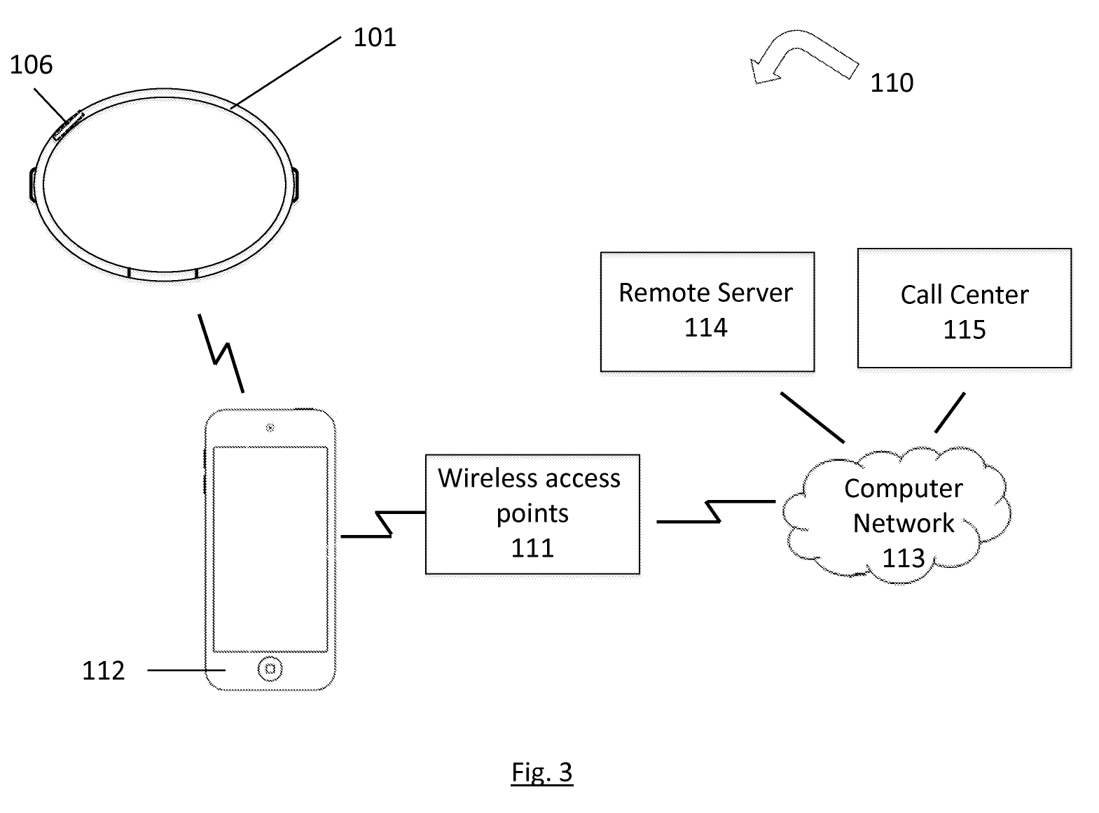

With reference to FIG. 3, there is shown an exemplary operating environment that comprises a communications system 110 that can be used to implement the methods disclosed herein. The communications system 110 generally includes the device 101, said device containing a wireless transmitting device 106. The communications system 110 also generally includes one or more wireless access points 111, a base station such as a smart phone 112, a computer network 113, a secure remote server 114 and a call center 115. The smart phone 112 can include computer processing capability, a transceiver capable of communicating using a wireless protocol, and a visual display screen 116. Examples of the smart phone 112 include the iPhone™ manufactured by Apple, Inc. and the Android™ manufactured by various companies. In another embodiment, the device communicates with a tablet, such as an iPad™ manufactured by Apple, Inc., instead of a smart phone. It should be understood that the disclosed method can be used with any number of different systems and is not specifically limited to the operating environment shown here.

The device 101 can be configured to communicate wirelessly with the base station 112 via the wireless transmitting device 106 according to one or more wireless protocols, such as any of the IEEE 802x protocols, radio, WiMAX, Wi-Fi, Li-Fi, LEDs or Bluetooth. In another embodiment, the device 101 may communicate to the base station 112 through a direct connection (e.g., USB connection). The data may also be transmitted from the device 101 to the base station 112 via audible transmissions. When the device 101 cannot communicate with the base station 112 via a wireless network (e.g., the device is too far from the smartphone) or via a direct connection (e.g., the device 101 is not connected to a base station 112 through a USB connection), the device 101 stores information in memory and continues to make gather data from the user. The device 101 automatically transmits all the stored information (along with a time/date stamp) to the base station 112 when the device 101 comes in proximity to a compatible wireless network.

After receiving data via wireless communication from a wireless transmitting device 106 on the device 101, the base station 112 may combine the received health data with environmental data collected from the base station 112, such as, but not limited to, location (via GPS technology), temperature, audio, video, and social networking status updates. The base station 112 combines this data with the processed data from the device 101, using a software application such as a mobile app. In addition, users may input additional data pertaining to health and wellbeing directly into the software application or mobile app on the base station 112. The software application/mobile app on the base station 112 will enable the user to monitor both health information collected by the device 101 and environmental information collected by the base station 112 directly on the visual display screen of the base station 112.

The base station 112 pushes the combined data, via the wireless protocols described above, over a computer network 113 to a secure remote server 114. The remote server 114 will collect, retain, store and display both historical and current health data from the device 101. The data is stored in a secure environment, with security levels equal to those of online banking and other confidential information. Conforming to Health Insurance Portability and Accountability Act (HIPAA) requirements, the system may employ data encryption employing logins, passwords, PINS and other verification. Pursuant to and compliant with HIPAA privacy regulations, a user's data may only be accessed on the remote server 114 by the user and any third parties explicitly authorized by the user to view the data. The remote server 114 provides an interface is accessible through an internet website. The interface displays vital information collected from the device 101 and the base station 112.

Operation

Referring back to FIG. 1 in conjunction with FIG. 3, the device 101 is worn by a user. When worn by a user, the one or more optical generators 102 and one or more optical sensors 103 are placed within close proximity of the skin. When the device 101 is in operation, the microprocessor 109 instructs the optical generator 102 to establish a communication link between the optical sensor 103 and the optical generator 102 by way of the reflective surface and a laser beam. The optical generator 102 then emits wavelengths of light. As noted above, the optical generator 102 may be a light emitting diode (“LED”) or solid-state laser. To prevent burning or irritation to the skin, the LED or laser will be at a low duty rate of modulation. The optical sensor 103 uses the wavelengths of light generated by the optical generator 102 to sense arterial pulsation and cutaneous pulsations and detect changes in the user's blood. As the heart pumps blood through a user's body, blood cells absorb and transmit varying amounts of the red and infrared radiation depending on how much oxygen binds to the cells' hemoglobin. The optical sensor 103 detects transmission at the predetermined wavelengths, for example red and infrared wavelengths. The optical sensor 103 provides the detected transmission to a pulse-oximetry circuit embedded within the device. For example, the optical sensor 103 can detect changes in the user's concentration of oxy- and deoxy-hemoglobin by observing their different respective spectra in the near-infrared range.

In another embodiment, the user applies his thumbs to both ECG/EKG sensor 107, thereby completing a circuit. The ECG/EKG sensors 107 non-invasively collect data from the user. In another embodiment, the device 101 includes an accelerometer. In another embodiment, the device 101 includes a mouthpiece into which the user may breathe. In another embodiment, the optical sensor 103 non-invasively collects data based on the user's sweat and the device 110 includes a pH sensitive dye which changes color when the pH of the sweat changes, as detected with a diffuse reflectance detector.

The data collected by the optical sensor 103 and/or ECG/EKG sensors 107 is digitized into a time-dependent optical waveform, which is then sent to the base station 112 through one or more protocols described above. The base station 112 collects and synchronizes data from the device 101 at predetermined intervals. The device 101 continues to detect and store data collected by the optical sensors 103 and/or ECG/EKG sensors 107 when not in close proximity to the base station 112. Once the device 101 is in close enough proximity to the base station 112, the device 101 automatically transmits all the stored data to the base station 112 via one or more of the protocols described above.

The base station 112 receives data from the device 101 via the wireless communication device 106 and downloads it to a software application, such as a mobile application, located on the base station 112. The software application may combine the received data with current environmental data collected by the base station 112, as described above. In addition, the user may input additional data pertaining to health (such as historical and/or current health data) directly into the software application.

The software application analyzes the processed data and generates health information. The generated health information may relate to, but not limited to: albumin levels; proteins; glucose; the blood's bicarbonate level; red blood cells; white blood cells; the user's hemoglobin concentration; blood volume; blood oxygenation levels; pulse rate (PR); blood pressure; saturation of peripheral oxygen (spO2); characteristics of hemoglobin (including but not limited to dehydration or hyperhydration; nitric oxide; anemia; carboxyhemoglobin; methemoglobin; oxygen content; oxygen saturation); the user's heart's electrical activity; the user's heartbeat; the user's risk of heart attack; pericarditis, angina; palpitations; thickness of the user's heart chambers' walls; the user's risk of stroke; sleep apnea; hypertension; the user's risk of congestive heart failure; atrial fibrillation; systolic embolism; the user's speed of movement; physiological changes during exercise (to include hydration and salt levels); the user's blood alcohol content; weight; body fat; lean mass; muscle mass; bone mass; body water; daily caloric intake; BMI; and visceral fat rating. The above-listed items are by no means an exhaustive list of all of the items that can be analyzed or measured by the device 101, but are simply an enumeration of some of the items that the device 101 is capable of analyzing or measuring.

The health information generated by the software application is transmitted, or pushed, to the remote server 114, with security levels consistent with the Health Insurance Portability and Accountability Act (HIPAA) requirements. The health information generated by the software application may be viewed on a visual display of the base station 112 and/or on the remote server 114. The user may input additional data pertaining to health (such as historical and/or current health data) directly into a website on the remote server 114. Information input into a website on the remote server 114 is transmitted either via a direct connection or wireless connection to the software application on the base station 112.

The software application on the base station 112, as well as a website on the remote serve 114, provide login screens for a user to enter user name and password, together with database credentials. After a user has entered his credentials, the user may review his health data in a graphical format and/or tabular format. The user can enter health-related data directly into the software application and/or website. Such health related data includes, but is not limited to, immunizations and dates; medications; physician names, addresses, phone numbers, email addresses; insurance information; emergency contact information; blood type; allergies; and medical history and diagnoses. A user's health data can be sorted and analyzed and the user can create reports for personal use or to provide to the user's physician.

The software application and/or website may include additional applications, information and links to additional websites based upon the user's individuals demographics and data. The software application and/or website may also include comparisons of a user's data to doctor-recommended values or may suggest that the user discuss the user's data with the user's physician. The software application and/or website may also include trends determined by applying mathematical and statistical rules (e.g. moving average and deviation) over a set of reading values. Trends are configurable by parameters that are either automatically calculated or are set by the user. The software application and/or website may use statistical methods or algorithms to identify potentially dangerous conditions. These algorithms range from the relatively simple (e.g., comparing blood pressure to a recommended value) to the complex (e.g., predictive medical diagnoses using ‘data mining’ techniques of historical data of multiple users). Examples of algorithms include, but are not limited to, an algorithm to check for sleep apnea, wherein the device 101 measures oxygen desaturations over a period of inactivity and an algorithm an algorithm for cardiac arrhythmias or irregularities, in which the application measures the length of time between each user pulse to determine the presence of a significant variation.

The software application and/or website may generate a warning or alert if the user's health information reflects a dangerous condition or a statistically significant aberration, judged by industry standards of health, based on an analysis of one or more data parameters using any type of algorithm. Alternatively, the message could be sent out when a data parameter (or multiple parameters) exceeds a predetermined value. The user chooses the destination where the alert may be sent. This destination may include e-mail, voicemail, text message, a call center 115 (discussed below) or any combination of the above.

With the user's consent, the user's health information stored on the secure remote server 114 may be accessed by authorized third parties, which may include, but is not limited to, the user's family, the user's medical team, hospitals, health insurance providers, pharmaceutical agencies conducting clinical trials, and other organizations. A third party may be assigned different access rights which may be less than the access rights of a user. With the user's consent, his health data may be forwarded to a database in the practicing physician's office, a health care management system or other health care facility or an insurance provider.

In another embodiment, the health information on the remote server 114 may be monitored by an authorized call center 115. The call center may be staffed with medical professionals such as doctors, nurses, or nurse practitioners. The call center may access the user's health data if the user has provided permission. If the user's health information reflects a dangerous condition, or the call center 115 determines whether the user is exhibiting symptoms of an emergency condition by polling vital user information generated by the device, the call center 115 contacts the emergency service providers in closest proximity to the user.

The invention allows a user to conduct low-cost, comprehensive, continuous monitoring of his health information. Data measured continuously throughout the day provides a relatively comprehensive data set compared to the data measured during infrequent and episodic medical appointments. This allows the user to monitor trends in the data that may indicate a medical condition.

What is claimed is:

1. A device for monitoring health information, said device comprising:
a material capable of being worn on a person's body;
an optical generator, wherein said optical generator is located on the interior of said material such that said optical generator is in close proximity to said person's skin;
an optical sensor, wherein said optical sensor is located on the interior of said material such that said optical sensor is in close proximity to said person's skin;
wherein said optical sensor is positioned diametrically opposite to said optical generator;
a processor; and
an interface for communicating with an external device.

2. The wearable device of claim 1, further comprising a computer memory.

3. The wearable device of claim 1, further comprising one or more of the following:
two EKG sensors; an accelerometer; a global positioning system (GPS) receiver;
a mouthpiece; a pH sensitive dye.

4. A health information monitoring system, comprising:
a wearable device comprising an optical generator, an optical sensor, a processor and an interface for communicating with an external device;
wherein said optical generator emits wavelengths of light;
wherein, in response to said emitted wavelengths of light, said optical sensor captures data;
wherein said processor processes said data captured by said optical sensor; and
wherein said interface transmits said processed data to said external device.

5. The system of claim 4, wherein said wearable device further comprises a computer memory and wherein said memory is configured to receive and store said processed data.

6. The system of claim 4, wherein said wearable device further comprises two EKG sensors.

7. The system of claim 4, wherein said wearable device further comprises one or more of the following: an accelerometer; a global positioning system (GPS) receiver; a mouthpiece; a pH sensitive dye.

8. A health information monitoring system, comprising:
a wearable device comprising an optical generator, an optical sensor, a processor and an interface for communicating with a call center;
wherein said optical generator emits wavelengths of light;
wherein, in response to said emitted wavelengths of light, said optical sensor captures data;
wherein said processor processes said data captured by said optical sensor; and
wherein said interface transmits said processed data to said call center.

9. The system of claim 8, wherein said call center monitors one of: the user's blood characteristics; the user's heart rate; the user's temperature; the user's blood pressure; the user's saturation of peripheral oxygen (spO2); the user's EKG measurements (including the user's risk of heart attack, pericarditis, angina, palpitations, congestive heart failure, atrial fibrillation and systolic embolism); the user's risk of stroke; sleep apnea; hypertension; the user's movement; and the user's respiratory rate.

10. A health information monitoring system, comprising:
a wearable device comprising an optical generator, an optical sensor, a processor and an interface for communicating with an external device;
wherein said optical generator emits wavelengths of light;
wherein, in response to said emitted wavelengths of light, said optical sensor captures data;
wherein said processor processes said data captured by said optical sensor;
wherein said interface transmits said processed data to said external device;
wherein said external device receives said processed data from said interface;
wherein said external device comprises a software application; and
wherein said software application comprises code to store, analyze and display said processed data.

11. The system of claim 10, wherein said software application is configured to receive environmental data collected by said external device, wherein said software application comprises code to store, analyze and display said environmental data in conjunction with said processed data.

12. The system of claim 10, wherein said wearable device further comprises a computer memory and wherein said memory is configured to receive and store said processed data.

13. The system of claim 10, wherein said wearable device further comprises two EKG sensors.

14. The system of claim 10, wherein said wearable device further comprises one or more of the following: an accelerometer; a global positioning system (GPS) receiver; a mouthpiece; a pH sensitive dye.

15. The system of claim 10, wherein said software application is configured to monitor one of: the user's blood characteristics; the user's heart rate; the user's temperature; the user's blood pressure; the user's saturation of peripheral oxygen (spO2); the user's risk of stroke and sleep apnea.

16. The system of claim 13, wherein said software application is configured to monitor one of: the user's EKG measurements (including the user's risk of heart attack, pericarditis, angina, palpitations, congestive heart failure, atrial fibrillation and systolic embolism); the user's risk of stroke; and the user's risk of hypertension.

17. The system of claim 14, wherein said software application is configured to monitor the user's movement.

18. The system of claim 14, wherein said software application is configured to monitor the user's respiratory rate.

19. A health information monitoring system, comprising:
a wearable device comprising an optical generator, an optical sensor, a processor and an interface for communicating with an external device;
wherein said optical generator emits wavelengths of light;
wherein, in response to said emitted wavelengths of light, said optical sensor captures data;
wherein said processor processes said data captured by said optical sensor;
wherein said interface transmits said processed data to said external device;
wherein said external device receives said processed data from said interface; and
wherein said processed data is transmitted from said external device to a remote server.

20. The system of claim 19, wherein said remote server comprises a website on which said processed data may be displayed, analyzed and stored.
